(19) United States
(12) Patent Application Publication
(10) Pub. No.: US 2008/0132444 A1
(43) Pub. Date: Jun. 5, 2008

(54) Ocular Agent Delivery Systems

(76) Inventors: Xiaoling Li, Danville, CA (US); Bhaskara Jasti, Stockton, CA (US); Ravichandran Mahalingam, Stockton, CA (US)

Correspondence Address: TECHNOLOGY & INTELLECTUAL PROPERTY;STRATEGIES GROUP PC (dba TIPS GROUP), P. O. BOX 1639, LOS ALTOS, CA, 94023-1639, US

(21) Appl. No.: 11/663,608

(22) PCT Filed: Sep. 29, 2005
(86) PCT No.: PCT/US05/35311

§ 371 Date: Jan. 30, 2008

Related U.S. Application Data
(60) Provisional application No. 60/617,453, filed on Oct. 9, 2004.

(51) Int. Cl. A61K 47/30; A61K 38/02; A61K 47/36; A61K 47/44; A61P 27/00; A61K 47/38; A61K 38/13

(52) U.S. Cl. 514/11; 514/772.3; 514/2; 514/781; 514/780; 514/777; 514/779

(57) ABSTRACT

This invention is directed to novel ocular agent delivery systems and, in particular, emulsions and molecular dispersions in the form of a gel comprising a hydrophobic ocular agent.

### BACKGROUND OF THE INVENTION

1. Field of Invention

This invention is directed to novel ocular agent delivery systems and, in particular, emulsions and molecular dispersions in the form of a gel comprising a hydrophobic ocular agent.

2. Description of the State of the Art

Current agent delivery systems used in the prevention, treatment, and ameliorization of systems of ocular disorders such as, for example, the use of conventional eye drops, are inefficient. Difficulties are most often associated with the solubility of the agent, the stability of the agent, and the ability to provide a sustained release. The terms “controlled release” and “sustained release” and “extended release” can be used interchangeably for purposes of the present invention and refer to an agent-containing formulation or a fraction thereof in which release of the agent is not an immediate release of the agent from the release medium. In some embodiments, the release can include a “delayed release.” In most embodiments, the term “sustained release” can be used in its conventional sense and refers to a formulation that provides for gradual release of an agent over an extended period of time.

Irrespective of the instilled volume, about 90-95% of the agent that is delivered using such conventional systems drains out of the conjunctival sac in 3-5 minutes due to continuous tear turnover, aqueous humor turnover, blinking, and nasolachrymal drainage. This translates into a poor precorneal residence time of agents in an ocular environment and necessitates frequent drug administration and increased dose intake. In addition, extensive drainage of agents through nasolachrymal ducts leads in systemic drug absorption and adverse effects.

Delivery systems based on hydrophilic gels have been proven to be effective in overcoming some of these limitations of conventional solutions or suspensions. These delivery systems can improve the precorneal residence of ophthalmic agents, improve the fraction of drug absorbed by the ocular tissues, and minimize the nasolachrymal drainage, systemic absorption of agents, and associated adverse effects.

Based on the gelling agent selected, as well as its strength, the release of agents from gels may also be sustained for prolonged periods. However, the capacity of hydrophilic gels for loading hydrophobic agents is limited. Under this circumstance, a large quantity of gel containing a limited quantity of drug needs to be applied to get the therapeutic benefits. Application of larger quantities of gels may result in blurring of vision and poor compliance. Alternative delivery systems such as hydrophilic emulsions (oil-in-water type) may be suitable to deliver such drug candidates.

Accordingly, one of skill in the art would appreciate a system designed to provide improved agent loading and delivery properties to the corneal surface. Such a system would serve as a promising step towards the management of external ocular diseases by providing enhanced solubility, stability, and sustained release of desired agents.

### SUMMARY

The present invention generally encompasses ocular agent delivery systems. More particularly, the systems of the present invention generally encompass emulsion-dispersed gels and molecular-dispersion gels for improving the loading and sustained-release of agents in an ocular environment for the treatment of an ocular disease.

In most embodiments, the invention includes a composition for treating ocular disease comprising a hydrophilic polymer, a hydrophobic ocular agent, and a gelling component, wherein the composition comprises a gel in an ocular environment and provides a sustained release of the hydrophobic ocular agent from the gel in the ocular environment. In some embodiments, the hydrophobic ocular agent comprises cyclosporine A, a protein, or a combination thereof. In some embodiments, the composition comprises a molecular dispersion of the hydrophobic ocular agent. In some embodiments, the composition of claim comprises an oil.

In many embodiments, the invention includes a method of forming an ocular gel comprising selecting an ocular agent for use in treating an ocular disease; mixing an oil in water to create an oil-in-water emulsion comprising the ocular agent and a non-ionic hydrophilic emulsifier; and combining the oil-in-water emulsion with a previously formulated gel comprising a gelling component. In some embodiments, the hydrophobic ocular agent comprises cyclosporine A, a protein, or a combination thereof.

In many embodiments, the invention includes a method of forming an ocular gel comprising selecting an ocular agent for use in treating an ocular disease; creating a molecular dispersion comprising the ocular agent and a hydrophilic polymer; and combining the molecular dispersion with a gel comprising a gelling component. In some embodiments, the hydrophobic ocular agent comprises cyclosporine A, a protein, or a, combination thereof. In some embodiments, the molecular dispersion comprises molecules, microparticles, controlled volumes, or a combination thereof.

In many embodiments, the invention includes a method of treating an ocular disease comprising administering an effective amount of the ocular delivery system taught herein to an ocular environment of a subject. In some embodiments, the ocular disease comprises keratoconjunctivitis sicca. In some embodiments, the method includes administering an additional agent to provide a combination therapy for the subject.

### BRIEF DESCRIPTION OF THE FIGURES

FIG. 1 is a scanning electron micrograph showing the physical stability of an oil phase containing a pharmaceutical agent in an E-Gel, according to some embodiments of the present invention.

FIG. 2 illustrates the in vitro release of CSA from an emulsion and from E-gels of varying agent concentrations, according to some embodiments of the present invention.

FIG. 3 illustrates the relationship between the mucoadhesion and viscosity of MD-Gels, according to some embodiments of the present invention.

FIG. 4 illustrates the comparative release profile of MD-Gels of varying gel strengths, according to some embodiments of the present invention.

### DETAILED DESCRIPTION

As discussed in more detail below, the embodiments of the present invention generally encompass ocular agent delivery systems. More particularly, the agents of the present invention generally encompass emulsion-dispersed gels and molecular-dispersion gels for improving the loading and sustained-release of agents in an ocular environment for the treatment of an ocular disease.

The present invention is directed to improving the bio-availability of agents, especially those agents whose activity is otherwise limited or eliminated because of their relative inability to be dissolved into aqueous media. The agents of most importance to the present invention are those which are either substantially insoluble in water or are not water-soluble to any appreciable degree. For example, a substantially insoluble agent can include those agents which are either non-soluble or only sparingly soluble in biological fluids, such as blood, lymph, gastrointestinal fluids, cerebrospinal fluid, plant saps, and the like.

In most embodiments, the agents are used for the treatment of ocular diseases, which include, but are not limited to, a disorder or pathological condition of the eye which is not normal to a subject in a healthy state.

In one embodiment, the ocular disease may be caused by a genetic defect. Examples of such ocular diseases for which a gene has been identified include, but are not limited to, autosomal retinitis pigmentosa, autosomal dominant retinitis punctual albescens, butterfly-shaped pigment dystrophy of the fovea, adult vitelliform macular dystrophy, Norrie's disease, blue cone monochromasy, choroideremia and gyrate atrophy. These may also be referred to as genetic ocular diseases.

In some embodiments, the ocular disease may not be caused by a specific known genotype (although they may be shown in the future to have a genetic component). These ocular diseases can include, but are not limited to, age-related macular degeneration, retinoblastoma, anterior and posterior uveitis, retinovascular diseases, cataracts, inherited corneal defects such as corneal dystrophies, retinal detachment and degeneration and atrophy of the iris, and retinal diseases which are secondary to glaucoma and diabetes, such as diabetic retinopathy.

In some embodiments, the ocular diseases include conditions that are not genetically based but still cause ocular disorders or disfunctions. These diseases include, but are not limited to, viral infections such as Herpes Simplex Virus or cytomegalovirus (CMV) infections, allergic conjunctivitis and other ocular allergic responses, dry eye, lysosomal storage diseases, glycogen storage diseases, disorders of collagen, disorders of glycosaminoglycans and proteoglycans, sphinogolipodoses, mucolipidoses, disorders of amino acid metabolism, dysthyroid eye diseases, antierior and posterior corneal dystrophies, retinal photoreceptor disorders, corneal ulceration and other ocular wounds such as those following surgery.

In some embodiments, an agent is insoluble if it has a solubility of less than 10 mg/mL in an aqueous medium having a pH ranging from about 1 to about 8. One of skill in the art can identify the solubilities of many substances, including drugs, by referring to reference source such as The Merck Index, 12th edition (1996), or through laboratory experiment. In some embodiments, an agent is insoluble if it has a solubility requiring at least 10,000 parts of an aqueous medium for 1 part of the agent.

Agent delivery systems are provided by the present invention to improve the properties of ocular agent delivery systems. Cyclosporine A (CSA), for example, is an immunomodulatory agent that is used in keratoconjunctivitis sicca, also known as dry-eye disease (DED). The poor aqueous solubility (6.6 μg/ml) of CSA limits its ability to form an aqueous solution or any other hydrophilic delivery system formulation. Studies have shown that topical treatments that include compositions having from about 0.05% to about 0.10% CSA can prevent T-cell activation. While not intending to be bound by any theory or mechanism of action, the prevention of T-cell activation can reverse inflammation of the ocular surface and lachrymal glands and mitigate the signs and symptoms of DED. Unfortunately, CSA can exhibit adverse effects when administered using conventional delivery techniques.

Some of the adverse effects of CSA include, but are not limited to, nephrotoxicity, hypertension, hepatotoxicity, changes in blood chemistry, and the like. Similarly, formulation into hydrophobic vehicles such as emulsions may increase the aqueous solubility, but these formulations are still limited by, for example, their poor mucoadhesion and corneal residence.

CSA has been incorporated into oily vehicles, emulsions, collagen shields and liposomes, and these methods of administration have been reported in the literature. All these systems have certain limitations, for example, limitations related to their stability and/or mode of application: oily vehicles may not improve the precorneal residence of agents over longer periods of time; emulsions are limited in that they are considered to be thermodynamically unstable systems; liposomes are associated with poor stability; and, collagen shields may impose difficulties during insertion and may include a foreign body sensation in the eyes. Treatments using other particulate carriers, such as nanosphere and microsphere suspensions, are generally associated with poor physical stability, limited precorneal residence time, and poor ocular availability.

Solubility enhancement techniques may improve the aqueous solubility of CSA, but it should be appreciated that formulating CSA into conventional delivery systems can still result in adverse effects. For example, the use of conventional ocular drops may again lead to poor corneal residence time and adverse systemic effects. Although gels can improve the corneal residence time, the poor aqueous solubility of some agents, such as CSA, remains a limitation to loading these agents into desired formulations.

Proteins can be particularly useful in the treatment of an ocular disease using the methods of the present invention. These ocular diseases may or may not have a genetic component. Examples of such ocular diseases include, for example, ocular wounds, allergies, viral infections, ulcerations, and the like. In some embodiments, gD is a protein that can be useful in the treatment of herpes simplex virus infections, transforming growth factor-β (TGF-β) can be used in treating corneal epithelial wounds; anti-IgE antibody can be used for treating ocular allergies, and brain derived neutrophic factor (BDNF) can be used for treating retinal degeneration. Neural growth factor (NGF) and neurotrophin 5 (NT5), as well as fusions and/or mutants of these, can be used for treating retinal degeneration or to delay or prevent damage after retinovasular disease, or retinal detachment or glaucoma. These neurotrophic factors may also be used to treat optic nerve compression, trauma, or demyelination. Immunosuppressive proteins may also be used to treat graft rejection after corneal transplantation. Vascular endothelial cell growth factor (VEGF) antagonists, such as antibodies or small molecules, may be used to treat neovascular disorders of the retina and vitreous. Basic fibroblast growth factor (bFGF) has been shown to prolong photoreceptor life.

In some embodiments, the improvements provided by the compositions of the present invention include improved solubility and sustained agent release, and these improvements can be particularly useful with regard to poorly soluble pharmaceutical agents in ophthalmic delivery systems. In some embodiments, a delivery system can include, for example, a pharmaceutical agent incorporated as an emulsion in a gel (E-Gel). In some embodiments, a delivery system can include, for example, a molecular dispersion of a pharmaceutical agent in a semisolid gel (MD-Gel). The present invention can provide benefits over conventional eye drops, emulsions, or gels; especially for the ocular administration of hydrophobic pharmaceutical agents to an ocular environment, such as a tear fluid. In some embodiments the tear fluid is in the ocular environment of a subject. In some embodiments, the ocular environment comprises artificial tear fluid in an in vitro ocular environment. In some embodiments, an artificial tear fluid is co-administered with the compositions of the present invention in the ocular environment of a subject.

E-Gels

The E-Gels of the present invention comprise an oily vehicle in a composition that includes a hydrophobic agent to enhance the solubility of the hydrophobic agent the composition. The emulsification of a hydrophobic agent using the methods of the present invention can provide a number of benefits. The benefits include, but are not limited to, the ability to (1) stabilize emulsions, (2) provide higher agent loadings, and (3) sustain the release of hydrophobic pharmaceutical agents in an ocular environment. In some embodiments, agent release from E-Gels can be sustained for longer than 1 hour, longer than 2 hours, longer than 3 hours, longer than 4 hours, longer than 5 hours, longer than 6 hours, longer than 10 hours, longer than 16 hours, or any range therein. In some embodiments, agent release can be sustained for 4-8 hours.

The E-Gels compositions of the present invention generally comprise an oil, an emulsifier, an agent comprising a hydrophobic agent, and a gelling agent. The method of preparing the E-Gels generally comprises preparing an emulsion of the oil, agent and emulsifier, and then gelling the emulsion with a gelling agent.

Any oil and emulsifier system known to one of skill in the art can be used in the present invention. In some embodiments, the oil comprises peanut oil. In some embodiments, the oil-to-water ratio can be about 0.5:100, 0.75:75, 1.0:50, 1.0:25, 1.0:10, 1:3, 1:5, or any ratio therein.

Any agent comprising a hydrophobic agent known to one of skill in the art can be used in the present invention. In some embodiments, the agent can comprise an ocular agent. In some embodiments, the agent comprises cyclosporine A. In some embodiments, the agent can comprise a combination of agents. In some embodiments, the agent can comprise a combination of agents including cyclosporine A.

Any gelling component known to one of skill in the art can be used in the present invention. In some embodiments, the gelling component can include, but are not limited to, modified celluloses such as hydroxypropylmethylcellulose, hydroxypropylethylcellulose, methylcellulose, sodium carboxymethylcellulose, and hydroxyethylcellulose, sodium alginate, alginic acid, tragacanth, polyacrylic acid and xanthan, guar, locust bean and karaya gums. In some embodiments, mixtures of two or more gelling components may also be used. In some embodiments, the gelling component can be, for example, a carboxyvinyl-polymer including, but not limited to, carboxypolymethylene.

In some embodiments, the viscosity of the gelling agent can be in the range of about 400 cp to about 2,000,000 cp. In some embodiments, the viscosity of the gelling agent can be within the range of about 1,000 cp to about 1,500,000 cp. In some embodiments, the viscosity of the gelling agent can be within the range of about 2,000 cp to about 100,000 cp.

In some embodiments, the gelling agent comprises CARBOPOL 934. In some embodiments, the concentration of the gelling agent can range from about 0.01% to about 5%, from about 0.05% to about 4%, from about 0.075% to about 3%, from about 0.1% to about 2.75%, from about 0.25% to about 2.5%, from about 0.5% to about 2.0%, or any range therein.

MD-Gels

The molecular dispersion gels (MD-Gels) of the present invention comprise particles of at least one component dispersed in another component, where the dispersion of the particles occurs on a molecular scale. In general, the compositions comprise a hydrophobic agent, a hydrophilic polymer, and a gelling component._In some embodiments, the MD-Gels can increase the incorporation capacity of hydrophobic agents into hydrophilic gelling systems and sustain their release. In some embodiments, the hydrophobic agent comprises CSA.

In some embodiments, agent release from MD-Gels can be sustained for longer than 1 hour, longer than 2 hours, longer than 3 hours, longer than 4 hours, longer than 5 hours, longer than 6 hours, longer than 10 hours, longer than 16 hours, or any range therein. In some embodiments, agent release can be sustained for 4-8 hours.

The agent can be either dispersed at a molecular level or dispersed as controlled volumes, such as microparticles. In some embodiments, the microparticles have an average particle size of less than about 1000 nm. In other embodiments, the microparticles have an average particle size of less than about 400 nm. Controlled volumes are discussed in more detail below. It should be appreciated that, in some embodiments, a molecular dispersion can include a range of physical states ranging from dispersions at the molecular level to amorphous or pre-crystalline associations of molecules, and even nanoparticle domains.

In some embodiments, the hydrophilic polymers include, but are not limited to, both biodegradable and non-biodegradable polymers such as, for example, poly(ethylene glycol)(PEG); poly(ethylene oxide); poly(ethylene glycol-co-propylene oxide)(PEG-PPO); dextran; dextrin; poly(vinyl alcohol); poly(methacrylates) such as poly((2-hydroxyethyl)methacrylate)(HEMA); poly(vinyl pyrrolidone); (PVP); poly(butylene terephthalate-co-ethylene glycol)(PBT-PEG or POLYACTIVE™); poly(alkylene oxalates); pluronic acid; sulfonated polystyrene; block copolymers with a bioabsorbable block and a perfluoro chain; PEG-caprolactone; PEG-D,L-lactide; biomolecules such as fibrin, fibrinogen, cellulose, starch, collagen, heparin and hyaluronic acid; poly(vinyl alcohols); and combinations thereof.

In some embodiments, the hydrophilic polymer comprises a poly(alkylene glycol). Examples of poly(alkylene glycols) include, but are not limited to, PEG, mPEG, poly(ethylene oxide), poly(propylene glycol)(PPG), poly(tetramethylene glycol), and any derivatives, analogs, homologues, congeners, salts, copolymers and combinations thereof. In some embodiments, the poly(alkylene glycol) is PEG. In other embodiments, the poly(alkylene glycol) is mPEG. In other embodiments, the poly(alkylene glycol) is poly(ethylene glycol-co-hydroxybutyrate).

In some embodiments, the hydrophilic moiety can be added in the range of from about 0.01% to about 99.99%; from about 0.1% to about 99.9%; from about 1% to about 99%; from about 3% to about 97%; from about 5% to about 95%; from about 7% to about 93%; from about 10% to about 90%; from about 15% to about 85%; from about 20% to about 80%; from about 25% to about 75%; from about 30% to about 70%; from about 40% to about 60%; about 50%; or any range therein, wherein the percent is a weight percent based on total polymer in the composition. It is to be appreciated that the range in which the hydrophilic moiety can be added depends upon the system design. It is to be appreciated that in some embodiments, any one or any combination of the hydrophilic, non-fouling polymers taught herein could excluded from any embodiment taught herein for reasons known to one of skill in the art.

The PEGs in all embodiments of the present invention can have molecular weights ranging from about 100 Daltons to about 100,000 Daltons, from about 200 Daltons to about 50,000 Daltons, from about 200 Daltons to about 20,000 Daltons, from about 300 Daltons to about 10,000 Daltons, from about 400 Daltons to about 9000 Daltons, from about 500 Daltons to about 5000 Daltons, from about 600 Daltons to about 2500 Daltons, or any range therein. It is to be appreciated that one skilled in the art should recognize that some of the groups, subgroups, and individual polymers may not be used in some embodiments of the present invention.

In some embodiments, the hydrophilic polymers have an intrinsic viscosity that can range from about 1,000 centipoise (cP) to about 3,000,000 cP, and a melt viscosity that can range from about 50 cP to about 100,000 cP. The viscosity of the polymer can be measured by a Brookfield Viscometer.

In some embodiments, the hydrophilic polymers have a pH-sensitive solubility. In some embodiments, the hydrophilic polymers can have a higher solubility in the pH range from about 1 to about 10. In some embodiments, the hydrophilic polymers can be more soluble at an acid pH than at neutral or basic pH. In some embodiments, the pH can be increased or decreased to achieve a desired solubility effect, such as gelation. In some embodiments, the MD-Gels can undergo rapid gelation upon raising the pH of the system, which improves the ease of administration of the dosage form while retaining the benefit of a higher drug loading and sustained the release. In some embodiments, the pH of the system can range from about 6.0 to about 9.0, from about 6.5 to about 8.5, from about 7.0 to about 8.0, from about 7.2 to about 7.8, or any range therein.

The molecular dispersions can be created in a variety of ways. In some embodiments, the dispersions can be created using flash-flow processing, wherein the hydrophilic polymer is subject to heat and shear to permit the agent to transfer into the morphology of the polymer matrix.

In some embodiments, the flash-flow processing can include either flash-heat processing or flash-shear processing. An example of a flash shear method is provided in U.S. Pat. No. 5,380,473 to Bogue et al., which is hereby incorporated herein by reference. In these embodiments, the temperature of a non-solubilized agent and hydrophilic polymer can be increased to a point at which the system undergoes internal-flow, followed by forcibly expelling or ejecting a stream of the polymer and agent, which provides a disruptive fluid shear force and separates the stream into separate masses having transformed morphology corresponding to the desired molecular dispersion.

In another embodiment, the system is subjected to a mix extrusion process, where a hydrophilic polymer and a hydrophobic agent are subjected to mixing under extrusion over a very short period of time, preferably not more than about two minutes, and most preferably not more than about thirty seconds, in order to obtain the desired molecular dispersion. Other methods of forming molecular dispersions are known to those skilled in the art and are within the scope of the present invention.

Ocular Agents

An ocular agent can comprise any agent useful in the treatment of ocular disease. In some embodiments, the ocular agents that can be used in the present invention include, but are not limited to, anti infective agents, such as amikacin, azithromycin, bacitracin, baquacil, cefazolin, chloramphenicol, chlorotetracycline, ciprofloxacin, clarithromycin, doxycycline, enfurvirtide, enoxacin, erythromycin, fleroxacin, fluconazole, ganciclovir, gatifloxacin, gentamicin, idoxuridine, levofloxacin, lomefloxacin, miconazole, moxifloxacin, natamycin, neomycin, norfloxacin, ofloxacin, polymyxin B, povidone iodine, propamidine, sparfloxacin, sulfacetamide, sulfisoxazole, tetracycline, tobramycin, trifluridine, vancomycin, vidarabine, and combinations thereof.

In some embodiments, the ocular agents that can be used in the present invention include, but are not limited to, anti-glaucoma agents, such as acetazolamide, alpha lipoic acid, apraclonidine, befunolol, betaxolol, bimatoprost, brimonidine, brimonidine, brinzolamide, bunazosin, carbachol, carteolol, demecarium, dipivefrin, dorzolamide, echothiophate, epinephrine, epinephrine, guanethidine, isofluorophate, isopropyl unoprostone, isosorbide, latanoprost, levobunolol, metipranolol, nipradilol, olvanil, physostigmine, pilocarpine, pindolol, propofol, puerarin, tetrahydrocannabinol, tilisolol, timolol, travoprost, unoptostone, and combinations thereof.

In some embodiments, the ocular agents that can be used in the present invention include, but are not limited to, anti-inflammatory agents such as aceclofanac, azapropazone, dexamethasone, etodolac, fenoprofen, flurbiprofen, fluorometholone, hydrocortisone, ibuprofen, ketoprofen, ketorolac, loteprednol etabonate, meclofenamate, mefenamic acid, methylprednisolone, nabumetone, naproxen, prednisolone, and combinations thereof.

In some embodiments, the ocular agents that can be used in the present invention include, but are not limited to, anti-allergics and decongestants such as antazoline, azelastine, cromolyn, emedastine, fexofenidine, ketorolac, ketotifen, levocabastine, lodoxamide, naphazoline, nedocromil, nephazoline, olopatadine, oxymetazolone, pemirolast, pheniramine, phenyleprine, tetrahydrozoline, and combinations thereof.

In some embodiments, the ocular agents that can be used in the present invention include, but are not limited to, immunomodulators such as azathioprine, chlorambucil, colchicine, cyclophosphamide, cyclosporine A, prednisolone, and combinations thereof.

In some embodiments, the ocular agents that can be used in the present invention include, but are not limited to, mydriatics such as atropine, cyclopentolate, homotropine, hydroxyamphetamine, phenyleprine, scopolamine, tropicamide, and combinations thereof.

In some embodiments, the ocular agents that can be used in the present invention include, but are not limited to, topical anesthetics such as benoxinate, cocaine, proparacaine, proxymetacaine, ropivacaine, tetracaine, and combinations thereof.

In some embodiments, the ocular agents that can be used in the present invention include, but are not limited to, ophthalmic dyes such as fluorescein, fluorexon, indocyanine green, rose bengal, and combinations thereof.

In some embodiments, the ocular agents that can be used in the present invention include, but are not limited to, calcitonin, cysteamine, diphoterine, fluorouracil, fluticasone, fomivirsen, lutein, mitomycin, pholedrine, pirenzepine, rhodamine 123, sulfamethoxypyridazine, verteporfin, vitamins, minerals and supplements, and combinations thereof.

One of skill in the art will appreciate that the agents taught above are merely provided by way of example and are not limiting with respect to the scope of the present invention. Many other agents known to one of skill in the art can be used. It should also be appreciated, however, that particular agents are unsuitable for particular uses and can be excluded from the scope of some embodiments. Furthermore, it should also be appreciated that each of the agents listed above can be administered as a derivative, an analog, a homolog, a congener, a salt, a prodrug, a codrug, a metabolite, an isomer, or a combination thereof.

Administration of Agents

The pharmaceutical compositions include an ocular agent in an amount that is diagnostic, therapeutic and/or prophylactic in the diagnosis, prevention, treatment and amelioration of symptoms of disease.

The terms “administration” or “administering” refer to a method of incorporating a compound into a subject's tissue, either ex vivo or in vivo to diagnose, prevent, treat, or ameliorate a symptom of a disease. In one example, a compound can be administered to a subject directly. The terms “subject” and “patient” can be used interchangeably and refer to an animal such as a mammal including, but not limited to, non-primates such as, for example, a cow, pig, horse, cat, dog, rat, and mouse; and primates such as, for example, a monkey or a human.

Combination Therapies

The agents of the present invention can be administered as a diagnostic, therapeutic or prophylactic agent in a combination therapy with the administering of one or more other agents. When the compound is incorporated in the subject in combination with one or active agents, the terms “administration” or “administering” can include sequential or concurrent incorporation of the compound with other agents.

The agents of the present invention can be administered concomitantly, sequentially, or cyclically to a subject. Cycling therapy involves the administering a first agent for a predetermined period of time, administering a second agent for a second predetermined period of time, and repeating this cycling for any desired purpose such as, for example, to enhance the efficacy of the treatment. The agents of the present invention can also be administered concurrently. The term “concurrently” is not limited to the administration of agents at exactly the same time, but rather means that the agents can be administered in a sequence and time interval such that the agents can work together to provide additional benefit. Each agent can be administered separately or together in any appropriate form using any appropriate means of administering the agent or agents. In some embodiments, the combination therapy provides a synergistic effect.

Each of the agents described herein can be administered to a subject in combination therapy according to a variety of regimens. In some embodiments, the agents can be administered at points in time that vary by about 15 minutes, 30 minutes, 1 hour, 2 hours, 4 hours, 8 hours, 12 hours, 18 hours, 24 hours, 48 hours or 1 week in time. In some embodiments, at least one of the agents is an immunomodulatory agent. In some embodiments, at least of the agents comprises CSA. In some embodiments, the agents can include antiproliferatives, antineoplastics, antimitotics, anti-inflammatories, antiplatelets, anticoagulants, antifibrins, antithrombins, antibiotics, antiallergics, antioxidants, and any prodrugs, codrugs, metabolites, analogs, homologues, congeners, derivatives, salts and combinations thereof.

The present invention encompasses sustained release formulations for each agent in the administration of one or more agents. In some embodiments, the sustained release formulations can reduce the dosage and/or frequency of the administrations of such agents to a subject.

Other agents that can be used in combination with the ocular agents of the present invention include, but are not limited to, bioactive agents such as antiproliferatives, antineoplastics, antimitotics, anti-inflammatories, antiplatelets, anticoagulants, antifibrins, antithrombins, antibiotics, antioxidants, and any prodrugs, metabolites, analogs, homologues, congeners, derivatives, salts and combinations thereof. The bioactive agents can include, for example, agonists and antagonists; small molecules; large molecules such as oligopeptides, polypeptides, proteins, oligonucleotides, polynucleotides, and combinations thereof. It is to be appreciated that one skilled in the art should recognize that some of the groups, subgroups, and individual bioactive agents may not be used in some embodiments of the present invention.

Antiproliferatives include, for example, actinomycin D, actinomycin IV, actinomycin I₁, actinomycin X₁, actinomycin C₁, dactinomycin (COSMEGEN®, Merck & Co., Inc.), imatinib mesylate, and any prodrugs, metabolites, analogs, homologues, congeners, derivatives, salts and combinations thereof. Antineoplastics or antimitotics include, for example, paclitaxel (TAXOL®, Bristol-Myers Squibb Co.), docetaxel (TAXOTERE®, Aventis S.A.), midostaurin, methotrexate, azathioprine, vincristine, vinblastine, fluorouracil, doxorubicin hydrochloride (ADRIAMYCIN®, Pfizer, Inc.) and mitomycin (MUTAMYCIN®, Bristol-Myers Squibb Co.), midostaurin, and any prodrugs, metabolites, analogs, homologues, congeners, derivatives, salts and combinations thereof.

Antiplatelets, anticoagulants, antifibrin, and antithrombins include, for example, sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, and thrombin inhibitors (ANGIOMAX®, Biogen, Inc.), and any prodrugs, metabolites, analogs, homologues, congeners, derivatives, salts and combinations thereof.

Cytostatic or antiproliferative agents include, for example, angiopeptin, angiotensin converting enzyme inhibitors such as captopril (CAPOTEN® and CAPOZIDE®, Bristol-Myers Squibb Co.), cilazapril or lisinopril (PRINIVIL® and PRINZIDE®, Merck & Co., Inc.); calcium channel blockers such as nifedipine; colchicines; fibroblast growth factor (FGF) antagonists, fish oil (omega 3-fatty acid); histamine antagonists; lovastatin (MEVACOR®, Merck & Co., Inc.); monoclonal antibodies including, but not limited to, antibodies specific for Platelet-Derived Growth Factor (PDGF) receptors; nitroprusside; phosphodiesterase inhibitors; prostaglandin inhibitors; suramin; serotonin blockers; steroids; thioprotease inhibitors; PDGF antagonists including, but not limited to, triazolopyrimidine; and nitric oxide; imatinib mesylate; and any prodrugs, metabolites, analogs, homologues, congeners, derivatives, salts and combinations thereof.

A pharmaceutical composition used to administer an agent is formulated to be compatible with its intended route of administration. Examples of routes of administration include, but are not limited to, parenteral such as, for example, intravenous, intradermal, intramuscular, and subcutaneous injection; oral; inhalation; intranasal; transdermal; transmucosal; and rectal administration.

In some embodiments, the administration can include oral administration of an agent, subcutaneous injection of an agent, intravenous injection using a sterile isotonic aqueous buffer, or a combination thereof. In some embodiments, the administration can include a solubilizing agent and a local anesthetic such as lignocaine to ease discomfort at the site of an injection. In some embodiments, the administrations can include any form of parenteral administration to obtain, for example, ease and uniformity of administration.

A “pharmaceutically acceptable carrier” for use in administering an agent can include a diluent, adjuvant, excipient, or vehicle with which an agent is administered. A carrier is pharmaceutically acceptable after approval by a state or federal regulatory agency or listing in the U.S. Pharmacopeial Convention or other generally recognized sources for use in subjects.

The pharmaceutical carriers include any and all physiologically compatible solvents, dispersion media, coatings, antibacterial and antifungal agents, isotonic and absorption delaying agents, and the like. Examples of pharmaceutical carriers include, but are not limited to, sterile liquids, such as water, oils and lipids such as, for example, phospholipids and glycolipids. These sterile liquids include, but are not limited to, those derived from petroleum, animal, vegetable or synthetic origin such as, for example, peanut oil, soybean oil, mineral oil, sesame oil, and the like. Water can be a preferred carrier for intravenous administration. Saline solutions, aqueous dextrose and glycerol solutions can also be liquid carriers, particularly for injectable solutions.

Suitable pharmaceutical excipients include, but are not limited to, starch, sugars, inert polymers, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition can also contain minor amounts of wetting agents, emulsifying agents, pH buffering agents, or a combination thereof. The compositions can take the form of solutions, suspensions, emulsion, tablets, pills, capsules, powders, sustained-release formulations and the like.

In some embodiments, an agent can be administered as an oral formulation and can include standard carriers such as, for example, pharmaceutical grades mannitol, lactose, starch, magnesium stearate, sodium saccharine, cellulose, magnesium carbonate, and the like. See Martin, E. W. Remington's Pharmaceutical Sciences. Supplementary active compounds can also be incorporated into the compositions.

In some embodiments, the carrier is suitable for any form of parenteral administration. In other embodiments, the carrier can be suitable for intravenous, intraperitoneal, intramuscular, sublingual or oral administration. In other embodiments, the pharmaceutically acceptable carrier may comprise pharmaceutically acceptable salts.

Pharmaceutical formulations for parenteral administration may include liposomes. Liposomes and emulsions are delivery vehicles or carriers that are especially useful for hydrophobic drugs. Depending on biological stability of the therapeutic reagent, additional strategies for protein stabilization may be employed. Furthermore, one may administer the drug in a targeted drug delivery system such as, for example, in a liposome coated with target-specific antibody. The liposomes can be designed to bind to a target protein and be taken up selectively by the cell expressing the target protein.

In some embodiments, any of the agents used in the present invention can be applied in controlled volumes to provide control over the concentration delivered, the rate of delivery, the surface chemistry relationship between the controlled volume and the matrix and, thus, the placement of the agent within the morphology of the matrix. The controlled volumes can all include droplets of pure agents, agents blended and/or conjugated with a polymer, agents encapsulated with a polymer, or a combination thereof, according to some embodiments of the present invention. These droplets can be formed using any source of pressure and application system known to one of skill in the art including, but not limited to, acoustic and piezoelectric pressure dispensing techniques.

The controlled-volumes can be delivered in a variety of sizes. In some embodiments, the controlled-volumes can be dispersed in volumes that range from about 1 femtoliter to about 1 microliter, from about 1 femtoliter to about 100 nanoliters, from about 1 femtoliter to about 10 nanoliters, from about 10 femtoliters to about 0.1 nanoliters, from about 10 femtoliters to about 100 picoliters, from about 100 femtoliters to about 10 picoliters, and any range therein. In some embodiments, the controlled-volume is smaller than 10 picoliters to assist in even distribution of monodisperse droplets. An advantage of this broad range of controlled-volume sizes is that agent delivery can be further controlled through control of the particle size.

Therapeutic compositions typically must be sterile and stable under the conditions of manufacture and storage. The composition can be formulated as a solution, microemulsion, liposome, or other ordered structure suitable for a high drug concentration. In some embodiments, the carrier can be a solvent or dispersion medium including, but not limited to, water; ethanol; a polyol such as for example, glycerol, propylene glycol, liquid polyethylene glycol, and the like; and, combinations thereof. The proper fluidity can be maintained in a variety of ways such as, for example, using a coating such as lecithin, maintaining a required particle size in dispersions, and using surfactants.

In some embodiments, isotonic agents can be used such as, for example, sugars; polyalcohols that include, but are not limited to, mannitol, sorbitol, glycerol, and combinations thereof; and sodium chloride. Sustained absorption characteristics can be introduced into the compositions by including agents that delay absorption such as, for example, monostearate salts, gelatin, and slow release polymers. Carriers can be used to protect active compounds against rapid release, and such carriers include, but are not limited to, controlled release formulations in implants and microencapsulated delivery systems.

Biodegradable and biocompatible polymers can be used such as, for example, ethylene vinyl acetate, polyanhydrides, polyglycolic acid, collagen, polyorthoesters, polylactic acid, polycaprolactone, polyglycolic copolymer (PLG), and the like. Such formulations can generally be prepared using methods known to one of skill in the art.

In some embodiments, any agent administered can be combined with one or more additional compounds that enhance the incorporation of the agent into a formulation of the present invention. Some compounds may be administered as suspensions such as, for example, oily suspensions for injection. Lipophilic solvents or vehicles include, but are not limited to, fatty oils such as, for example, sesame oil; synthetic fatty acid esters, such as ethyl oleate or triglycerides; and liposomes. Suspensions that can be used for injection may also contain substances that increase the viscosity of the suspension such as, for example, sodium carboxymethyl cellulose, sorbitol, or dextran. Optionally, a suspension may contain stabilizers or agents that increase the solubility of the compounds and allow for preparation of highly concentrated solutions.

In some embodiments, a sterile and injectable solution can be prepared by incorporating an effective amount of an active compound in a solvent with any one or any combination of desired additional ingredients described above, filtering, and then sterilizing the solution. In some embodiments, dispersions can be prepared by incorporating an active compound into a sterile vehicle containing a dispersion medium and any one or any combination of desired additional ingredients described above. Sterile powders can be prepared for use in sterile and injectable solutions by vacuum drying, freeze-drying, or a combination thereof, to yield a powder that can be comprised of the active ingredient and any desired additional ingredients. Moreover, additional ingredients can be from a separately prepared sterile and filtered solution.

In some embodiments, agents can be administered by inhalation through an aerosol spray or a nebulizer that may include a suitable propellant such as, for example, dichlorodifluoromethane, trichlorofluoromethane, dichlorotetrafluoroethane, carbon dioxide, or a combination thereof. In one example, a dosage unit for a pressurized aerosol may be delivered through a metering valve. In another example, capsules and cartridges of gelatin, for example, may be used in an inhaler and can be formulated to contain a powderized mix of the compound with a suitable powder base such as, for example, starch or lactose.

Selecting an Amount of a gent to be Delivered

The amount of an agent to be delivered in the compositions can vary according to factors such as the type of agent, type of disease, age, sex, and weight of the subject. Dosage regimens may be adjusted to optimize a therapeutic response. In some embodiments, a single dose may be administered; several divided doses may be administered over time; the dose may be proportionally reduced or increased; or any combination thereof, as indicated by the exigencies of the therapeutic situation and factors known one of skill in the art.

It is to be noted that dosage values may vary with the severity of the condition to be alleviated. Dosage regimens may be adjusted over time according to the individual need and the professional judgment of the person administering or supervising the administration of the compositions, and the dosage ranges set forth herein are exemplary only and do not limit the dosage ranges that may be selected by medical practitioners.

An “effective amount” of a compound of the invention can be used to describe a therapeutically effective amount or a prophylactically effective amount. A “therapeutically effective amount” refers to an amount that is effective at the dosages and periods of time necessary to achieve a desired therapeutic result and may also refer to an amount of active compound, prodrug or pharmaceutical agent that elicits any biological or medicinal response in a tissue, system, or subject that is sought by a researcher, veterinarian, medical doctor or other clinician that may be part of a treatment plan leading to a desired effect.

In some embodiments, the therapeutically effective amount may need to be administered in an amount sufficient to result in amelioration of one or more symptoms of a disorder, prevention of the advancement of a disorder, or regression of a disorder. In one example, treatment of an inflammatory disorder or an autoimmune disorder characterized by inflammation, a therapeutically effective amount preferably refers to the amount of a therapeutic agent that reduces the inflammation of tissue by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. The term “treating” refers to the administering one or more diagnostic, therapeutic or prophylactic agents including, but not limited to, ocular agents.

A “prophylactically effective amount” refers to an amount that is effective at the dosages and periods of time necessary to achieve a desired prophylactic result. Typically, a prophylactic dose is used in a subject prior to the onset of a disease, or at an early stage of the onset of a disease, to prevent or inhibit onset of the disease or symptoms of the disease. A prophylactically effective amount may be less than, greater than, or equal to a therapeutically effective amount.

An agent of the present invention can be administered in a dosage unit. The term “dosage unit” refers to discrete, predetermined quantities of a compound that can be administered as unitary dosages to a subject. A predetermined quantity of active compound can be selected to produce a desired therapeutic effect and can be administered with a pharmaceutically acceptable carrier. The predetermined quantity in each unit dosage can depend on factors that include, but are not limited to, (a) the unique characteristics of the active compound and the particular therapeutic effect to be achieved, and (b) the limitations inherent in the art of creating and administering such dosage units.

In some embodiments, a therapeutically or prophylactically effective amount of an agent may range in concentration from about 0.001 nM to about 0.1 M; from about 0.001 nM to about 0.05 M; from about 0.01 nM to about 15 μM; from about 0.01 nM to about 10 μM, or any range therein. In some embodiments, the agent may be administered in an amount ranging from about 0.001 mg/kg to about 50 mg/kg; from about 0.005 mg/kg to about 40 mg/kg; from about 0.01 mg/kg to about 30 mg/kg; from about 0.01 mg/kg to about 25 mg/kg; from about 0.1 mg/kg to about 20 mg/kg; from about 0.2 mg/kg to about 15 mg/kg; from about 0.4 mg/kg to about 12 mg/kg; from about 0.15 mg/kg to about 10 mg/kg, or any range therein, wherein a human subject, for example, is assumed to average about 70 kg.

In some embodiments, the agent is CSA, and the delivery system is a topical ocular delivery system having from about 0.005% to about 0.50% CSA (w/w). In some embodiments, the topical ocular delivery system has from about 0.01% to about 0.40% CSA, from about 0.025% to about 0.30% CSA, from about 0.03% to about 0.25% CSA, from about 0.04% to about 0.20% CSA, from about 0.05% to about 0.15% CSA, from about 0.075% to about 0.125% CSA, from about 0.08% to about 0.10% CSA, or any range therein.

Articles of Manufacture

The present invention provides for articles of manufacture that encompass finished, packaged and labelled pharmaceutical products. The articles of manufacture include a pharmaceutical formulation in an appropriate vessel or container such as, for example, a tube, a vial, a delivery apparatus such as a syringe, or any other container that is hermetically sealed. In the case of dosage forms suitable for parenteral administration, the active ingredient, e.g. one or more agents including an ocular agent, is sterile and suitable for administration. In other words, the invention can encompass both parenteral solutions and lyophilized powders, each being sterile, and the latter being suitable for reconstitution prior to injection. Alternatively, the unit dosage form may include a solid suitable for oral, transdermal, topical or mucosal delivery.

In some embodiments, an agent can be provided in a unit dosage form that is suitable for intravenous, intramuscular, topical or subcutaneous delivery. Thus, the invention encompasses solutions, which are preferably sterile and suitable for each route of delivery. The concentration of agents and amounts delivered are included as described herein.

As with any pharmaceutical product, the packaging material and container are designed to protect the stability of the product during storage and shipment. In addition, the articles of manufacture can include instructions for use or other information material that can advise the user such as, for example, a physician, technician or patient, regarding how to properly administer the composition as a diagnostic, prophylactic, therapeutic, or ameliorative treatment of the disease of concern. In some embodiments, instructions can indicate or suggest a dosing regimen that includes, but is not limited to, actual doses and monitoring procedures.

In some embodiments, the instructions can include informational material indicating that the administering of the compositions can result in adverse reactions including but not limited to allergic reactions such as, for example, anaphylaxis. The informational material can indicate that allergic reactions may exhibit only as mild pruritic rashes or may be severe and include erythroderma, vasculitis, anaphylaxis, Steven-Johnson syndrome, and the like. The informational material should indicate that anaphylaxis can be fatal and may occur when any foreign substance is introduced into the body; and, that these allergic reactions can manifest themselves as urticaria or a rash and develop into lethal systemic reactions and can occur soon after exposure such as, for example, within 10 minutes. The informational material can further indicate that an allergic reaction may cause a subject to experience paresthesia, hypotension, laryngeal edema, mental status changes, facial or pharyngeal angioedema, airway obstruction, bronchospasm, urticaria and pruritus, serum sickness, arthritis, allergic nephritis, glomerulonephritis, temporal arthritis, eosinophilia, or a combination thereof.

In some embodiments, the articles of manufacture can comprise one or more packaging materials such as, for example, a box, bottle, tube, vial, container, sprayer, insufflator, intravenous (I.V.) bag, envelope, and the like; and at least one formulation comprising an ocular agent, such as CSA, within the packaging material. In other embodiments, the articles of manufacture may also include instructions for using the composition as a diagnostic, prophylactic, therapeutic, or ameliorative treatment for the disease of concern.

In some embodiments, the articles of manufacture can comprise one or more packaging materials such as, for example, a box, bottle, tube, vial, container, sprayer, insufflator, intravenous (I.V.) bag, envelope, and the like; and a first composition comprising at least one formulation comprising an ocular agent, such as CSA, within the packaging material, along with a second composition comprising a second agent such as, for example, a glycosaminoglycan, phospholipid, poly(alkylene glycol), any other bioactive agent taught herein, or any prodrugs, codrugs, metabolites, analogs, homologues, congeners, derivatives, salts and combinations thereof.

### EXAMPLES

The following examples have been provided to teach some of the concepts and embodiments set forth herein and are in no way meant to be limiting to the scope of present invention.

### Example 1

Formation of an E-Gel

CSA was incorporated in hydrophilic emulsions having 1:1, 1:3, 1:5 and 1:10 ratios of oil (i.e. peanut oil) to water and emulsified using a non-ionic hydrophilic emulsifier (i.e. polyoxyethylene sorbitan monooleate; Polysorbate 80). The dispersion was stirred at 2500 rpm using a stirrer for 30 minutes and the emulsions were characterized for the globule size and zeta potential. The content of pharmaceutical agent was determined using a reversed phase HPLC method.

The emulsions were dispersed into previously formulated and neutralized gels containing 0.25, 0.50, 0.75, 1.00 and 1.50% (w/w) of a gelling component (i.e. prop-2-enoic acid; CARBOPOL 934) in a ratio of emulsion to gel of about 1:10 to form the E-Gels. When the emulsion was incorporated into gels, they did not cream or coalesce, indicating a significant improvement in their physical stability. The physical stability of E-Gels was evaluated by microscopic method and the loading of pharmaceutical agent was determined after extracting the drug with methanol and quantifying the amount using high pressure liquid chromatography (HPLC).

A loading of 8 mg/mL of the CSA was achieved in the emulsion, which is a significantly high amount when compared to CSA's aqueous solubility. All emulsions separated within 12 h period, which may be due to the large differences in the densities of the peanut oil and water media. It was observed that all emulsions containing the pharmaceutical agent were milky white in color and free flowing.

TABLE 1 shows the globule size and zeta potential of emulsions containing different phase-volume ratio. The size of the dispersed phase was measured using an optical microscope and a scanning electron microscope. The mean globule size of the internal phase ranged from 1.34 to 2.19 μm and the zeta potential from −24.4 to −34.7 mV.

TABLE 1

| | Phase volume ratio (peanut oil:water) | | | |
|---|---|---|---|---|
| Description | 1:1 | 1:3 | 1:5 | 1:10 |
| Globule | 1.34 ± 0.49 | 1.42 ± 1.20 | 1.49 ± 0.59 | 2.19 ± 0.33 |
| size (μm) | | | | |
| Zeta | −24.4 ± 0.3 | −34.7 ± 3.6 | −31.6 ± 3.7 | −30.2 ± 2.0 |
| potential (mV) | | | | |

At all the phase volume ratios studied, the globule size remained constant, while the zeta potential remained the same at 1:3, 1:5 and 1:10 ratios. However, emulsions at 1:5 and 1:10 phase volume ratio were found more stable (visual observation) and hence 1:5 ratio was used to incorporate in the gels.

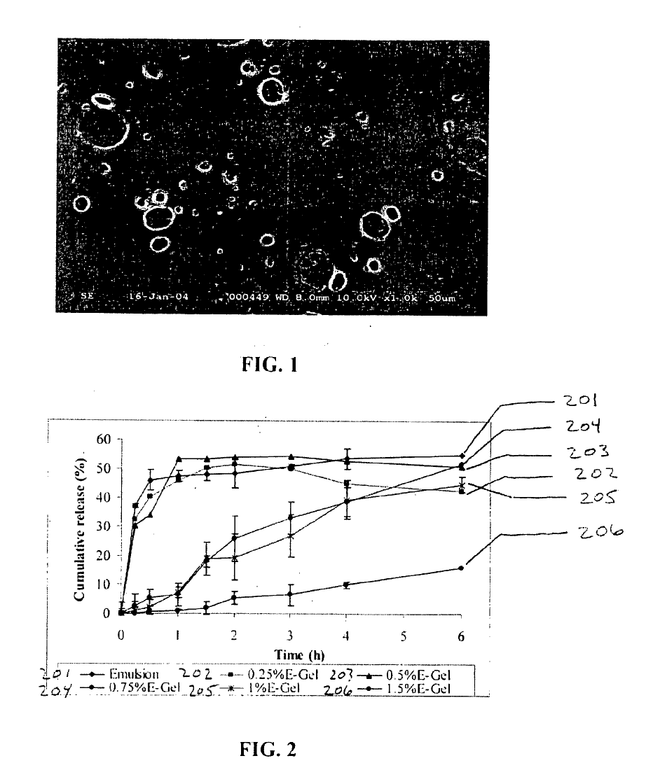

FIG. 1 is a scanning electron micrograph showing the physical stability of an oil phase containing a pharmaceutical agent in an E-Gel, according to some embodiments of the present invention. Microscopic examination of these preparations showed that the emulsion remained dispersed when stored at room temperature for 4 weeks. No significant change in the content of pharmaceutical agent was observed during this period.

The release of pharmaceutical agent from these systems was studied in artificial tear fluid maintained at 37±1° C. and stirred at 20 rpm. Aliquots of 1 mL were withdrawn at different time intervals over 6 hours and analyzed for agent content. FIG. 2 illustrates the in vitro release of CSA from an emulsion and from E-gels of varying gelling component concentrations, according to some embodiments of the present invention. In the release studies, the emulsion 201 and two E-Gels—one having 0.25% agent 202 and one having 0.50% agent 203—exhibited an initial burst release ranging from 30 to 37% within 0.25 hours. The E-Gels having 0.75% agent 204, 1.0% agent 205, and 1.5% agent 206 were effective at prolonging agent release of CSA for over 6 hours.

Accordingly, release rate was dependent on the gelling component's concentration. The time for 50% release (T50 dissolution) of pharmaceutical agent from the emulsion, 0.25 and 0.50% w/w E-Gels were within 3 hours, whereas, it was about 6 hours in case of 0.75% w/w E-Gel. The T50 of 1.00% and 1.50% (w/w) E-Gels were further extended. The release of pharmaceutical agent from these systems was studied using a modified USP type 2 apparatus.

### Example 2

Formation of an MD-Gel

About 2.5 g of hydrophilic carrier (PEG 1350) was placed in a glass test tube and melted in a hot water bath. When it formed a clear solution, 25 mg of the pharmaceutical agent (CSA) was added and stirred until dissolved to form a uniform colloidal molecular dispersion (MD).

Aqueous dispersions containing 0.3%, 0.5%, and 0.7% (w/v) of the gelling component (CARBOPOL 934) were prepared by sprinkling the gelling component over purified water under stirring. These three dispersions (coded as G1, G2 and G3) were allowed to hydrate overnight and the contents were stirred to homogenize the systems. About 1.2 g MD was added to about 10.8 g each of G1, G2 or G3 and neutralized with sodium hydroxide to form MD-Gels. The MD-Gels were coded as MDG1, MDG2 and MDG3. These systems were evaluated for their rheologic properties, mucoadhesive properties, loading capacity, and in vitro release of pharmaceutical agent.

The solubility of pharmaceutical agents could be enhanced, in some embodiments, by up to 182 fold using this molecular dispersion approach. TABLE 2 shows the concentration of agent in the MD-Gels. The agent concentration was determined using an optimized HPLC method. From the reported aqueous CSA solubility of 6.6 μg/ml, the solubility enhancement of the CSA in the molecular dispersion was about 182 fold.

TABLE 2

| Description | Solubility (μg/ml) | Enhancement factor |
|---|---|---|
| Pharmaceutical agent | 6.6 | 1 |
| Molecular dispersions of | 1200 | 182 |
| pharmaceutical agent | | |

TABLE 3 shows the viscosity of the formulations. A Brookfield Synchrolectric viscometer (RVT model) was used for the theological characterization. The viscosity of the gel forming solution (before neutralization) was measured with #2 spindle at 6 rpm for purposes of comparative evaluations. The rheology of MD-Gels was studied using a T bar spindle and a constant spindle/speed factor F, where measurements at 0.3 rpm were used for comparative evaluation.

TABLE 3

| | Gel forming solution | | MD-Gels | |
|---|---|---|---|---|
| Gelling | (before neutralization) | | (after neutralization) | |
| component | | Viscosity | | Viscosity |
| (%) | Formulation | (cp) | Formulation | (cp) |
| 0.3 | G1 | 125 | MDG1 | 1,132,200 |
| 0.5 | G2 | 750 | MDG2 | 1,265,400 |
| 0.7 | G3 | 2,392 | MDG3 | 1,448,550 |

The agents can be successfully formulated into drops that are pH-triggered to form gels in situ through the use of a gelling component. The gel forming solutions exhibited a low viscosity, but as the pH was increased to 7.4 (the pH of the tear fluid), the solutions transformed into high viscosity gels. This in situ physical transformation in consistency, from sol to gel, provides a method for an easier administration of agent that can be accompanied by a prolonged precorneal residence time.

### Example 3

Mucoadhesive Properties of an MD-Gel

The MD-Gels retain their mucoadhesive properties. Moreover, the mucoadhesive strength of MD-Gels gels increases with an increase in the content of gelling component. A good correlation between mucoadhesion and viscosity has been observed, where an r² value of 0.9623 was obtained in a least squares linear regression analysis of mucoadhesive strength as a function of viscosity.

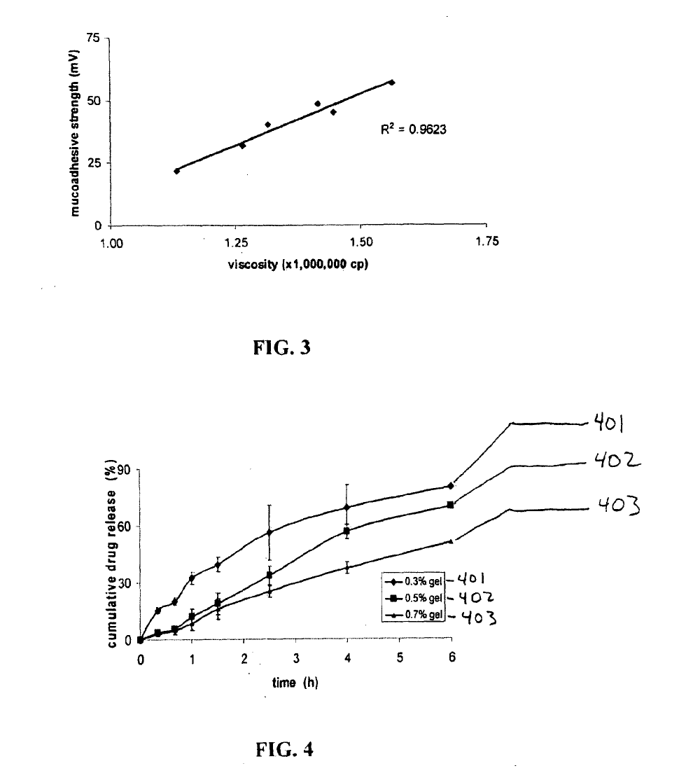

FIG. 3 illustrates the relationship between the mucoadhesion and viscosity of MD-Gels, according to some embodiments of the present invention. Fresh Porcein eyelid tissues were collected for mucoadhesive strength studies. An empty syringe was taken and a 0.6 mm hole was made at the top of the barrel. About 1.5×1.5 mm eye mucosal tissue was separated for studies and placed on the piston of the syringe and pushed to the top of the barrel to get about 0.5 mm diameter tissue projecting out of the barrel. The assembly was on the mucoadhesive strength tester with the load cell. The MD-Gel was applied to the tissue surface and placed on the bottom metallic blank. After allowing the arrangement to settle for one minute, force was applied to separate the tissue from the gel.

### Example 4

Agent Release Properties of an MD-Gel

Glass bottles were mounted on a shaker on a temporary base. About 0.5 g of the sample gel was carefully placed at the bottom of the glass bottle. A measured volume of 10 ml of freshly prepared artificial tear fluid (ATF) was placed in the bottle carefully without disturbing the gel, and the bottle was capped.

The composition of artificial tear fluid used was 0.670 g NaCl, 0.200 g NaHCO₃, and 0.008 g CaCl₂.2H₂O, with purified water in a quantity sufficient (q.s.), where the q.s. was 100 g in this example. The shaker was rotated at 80 rpm; and, 1 ml aliquots were withdrawn at 20 min, 40 min, 1 h, 1.5 h, 2.5 h, 4 h, and 6 h intervals and replaced by an equal volume of the receptor medium. The aliquots were analyzed using high pressure liquid chromatography (HPLC) at 210 nm.

FIG. 4 illustrates the comparative release profile of MD-Gels of varying gel strengths, according to some embodiments of the present invention. The MD-Gel comprised of 0.3% gelling component 401 released more agent at a given period of time than the MD-Gel comprised of 0.5% gelling component 402. Likewise, each of the MD-Gels 401 and 402 released more agent at a given period of time than the MD-Gel comprised of 0.7% gelling component 403. In sum, each of the MD-gels provided a sustained release of pharmaceutical agent over at least 6 hour period.

We claim:

1. A composition for treating ocular disease comprising a hydrophilic polymer, a hydrophobic ocular agent, and a gelling component, wherein the composition comprises a gel in an ocular environment and provides a sustained release of the hydrophobic ocular agent from the gel in the ocular environment.

2. The composition of claim 1, wherein the hydrophobic ocular agent comprises cyclosporine A, a protein, or a combination thereof.

3. The composition of claim 1 comprising a molecular dispersion of the hydrophobic ocular agent.

4. The composition of claim 1, wherein the hydrophilic polymer comprises a poly(alkylene glycol) or a non-ionic hydrophilic emulsifier.

5. The composition of claim 4, wherein the poly(alkylene glycol) comprises poly(ethylene glycol).

6. The composition of claim 4, wherein the non-ionic hydrophilic emulsifier comprises polyoxyethylene sorbitan monooleate.

7. The composition of claim 1, wherein the gelling component comprises a component selected from a group consisting of hydroxypropylmethylcellulose, hydroxypropylethylcellulose, methylcellulose, sodium carboxymethylcellulose, and hydroxyethylcellulose, sodium alginate, alginic acid, tragacanth, polyacrylic acid, xanthan gum, guar gum, locust bean gum, karaya gum carboxyvinyl polymers, and combinations thereof.

8. The composition of claim 7, wherein the gelling component comprises carboxypolymethylene.

9. The composition of claim 1 comprising an oil.

10. The composition of claim 9, wherein the oil comprises peanut oil.

11. A method of forming an ocular gel comprising:
selecting an ocular agent for use in treating an ocular disease;
mixing an oil in water to create an oil-in-water emulsion comprising the ocular agent and a non-ionic hydrophilic emulsifier;
combining the oil-in-water emulsion with a previously formulated gel comprising a gelling component selected from a group consisting of hydroxypropylmethylcellulose, hydroxypropylethylcellulose, methylcellulose, sodium carboxymethylcellulose, and hydroxyethylcellulose, sodium alginate, alginic acid, tragacanth, polyacrylic acid, xanthan gum, guar gum, locust bean gum, karaya gum carboxyvinyl polymers, and combinations thereof.

12. The method of claim 11, wherein the hydrophobic ocular agent comprises cyclosporine A, a protein, or a combination thereof.

13. The method of claim 11, wherein the non-ionic hydrophilic emulsifier comprises polyoxyethylene sorbitan monooleate.

14. The method of claim 11, wherein the gelling component comprises carboxypolymethylene.

15. The method of claim 11, wherein the oil comprises peanut oil.

16. A method of forming an ocular gel comprising:
selecting an ocular agent for use in treating an ocular disease;
creating a molecular dispersion comprising the ocular agent and a hydrophilic polymer;
combining the molecular dispersion with a gel comprising a gelling component selected from a group consisting of hydroxypropylmethylcellulose, hydroxypropylethylcellulose, methylcellulose, sodium carboxymethylcellulose, and hydroxyethylcellulose, sodium alginate, alginic acid, tragacanth, polyacrylic acid, xanthan gum, guar gum, locust bean gum, karaya gum, carboxyvinyl polymers, and combinations thereof.

17. The method of claim 16, wherein the hydrophobic ocular agent comprises cyclosporine A, a protein, or a combination thereof.

18. The method of claim 16, wherein the molecular dispersion comprises molecules, microparticles, controlled volumes, or a combination thereof.

19. The method of claim 16, wherein the hydrophilic polymer comprises a component selected from a group consisting of poly(ethylene glycol) (PEG); PEG-caprolactone; PEG-D,L-lactide; poly(ethylene glycol-co-propylene oxide); poly(vinyl alcohol); poly((2-hydroxyethyl)methacrylate); poly(vinyl pyrrolidone); poly(butylene terephthalate-co-ethylene glycol); poly(alkylene oxalates); poly(vinyl alcohols); pluronic acid; sulfonated polystyrene; dextran; dextrin; fibrin, fibrinogen, cellulose, starch, collagen, heparin and hyaluronic acid; and combinations thereof.

20. The method of claim 16, wherein the gelling component comprises carboxypolymethylene.

21. A method of treating an ocular disease comprising administering an effective amount of the composition of claim 1 to an ocular environment of a subject.

22. The method of claim 21, wherein the ocular disease comprises keratoconjunctivitis sicca.

23. The method of claim 21 further comprising administering an additional agent to provide a combination therapy for the subject.
